(19) United States
(12) Patent Application Publication
(10) Pub. No.: US 2020/0268545 A1
(43) Pub. Date: Aug. 27, 2020

(54) BRACE-TYPE ANTI-SNORING DEVICE

(71) Applicant: Ching-Yi Huang, Taoyuan City 320 (TW)

(72) Inventor: Ching-Yi Huang, Taoyuan City 320 (TW)

(21) Appl. No.: 16/282,557

(22) Filed: Feb. 22, 2019

(51) Int. Cl. A61F 5/56

(52) U.S. Cl. CPC A61F 5/566; A61F 2005/563

(57) ABSTRACT

A brace-type anti-snoring device includes a base and a flow-guide conduit. The base is placed within an oral cavity, palate teeth occlude an upper occlusal channel of the base, and mandible teeth occlude a lower occlusal channel of the base. Meanwhile, the flow-guide conduit is disposed outside the oral cavity. Thus, after the user has closed lips and swallowed saliva, a negative pressure difference is generated in the oral cavity and a tongue is sucked frontward and obstructs an air intake hole of the base concurrently to open the respiratory tract and fix the tongue and to achieve the objective of preventing snoring.

### BACKGROUND OF THE INVENTION

### (1) Field of the Invention

The invention relates to a brace-type anti-snoring device, and more particularly to a device structure capable of preventing snoring and capable of opening the respiratory tract to prevent the respiratory tract from being obstructed.

### (2) Description of the Prior Art

At present, many people snore as they sleep. In the past, it is considered that snoring is the micropathy, which is normal and harmless to the health. However, with the development and advancement of medicine, it is found that snoring is due to respiratory tract obstruction accompanying apnea or gasp, and is also referred to as the sleep apnea. The sleep apnea may affect the sleep quality, or even induce the illness, such as the high blood pressure, heart disease or the like. In order to solve the snoring problem, there are many products, such as the so-called snoring stoppers or anti-snoring devices, available on the market. At present, however, the commercially available snoring stopper or anti-snoring device still cannot really and effectively solve the problem of the respiratory tract obstruction, and can only slightly ease the symptom, and many snoring stoppers or anti-snoring devices still have considerable operation inconvenience upon actual use.

### SUMMARY OF THE INVENTION

A main objective of the invention is to provide a brace-type anti-snoring device, which is mainly constituted by a base and a flow-guide conduit. The base is placed within an oral cavity, palate teeth occlude an upper occlusal channel of the base, and mandible teeth occlude a lower occlusal channel of the base. Meanwhile, the flow-guide conduit is disposed outside the oral cavity. Thus, after the user has swallowed saliva, a negative pressure difference is generated in the oral cavity and a tongue is sucked frontward and obstructs an air intake hole of the base concurrently to open the respiratory tract and fix the tongue and to achieve the objective of preventing snoring.

Further aspects, objects, and desirable features of the invention will be better understood from the detailed description and drawings that follow in which various embodiments of the disclosed invention are illustrated by way of examples.

### BRIEF DESCRIPTION OF THE DRAWINGS

FIG. 1 is a pictorial outlook view of the invention.

FIG. 2 is a cross-sectional view of the invention.

FIG. 3 is a first pictorially exploded view of the invention.

FIG. 4 is a second pictorially exploded view of the invention.

FIG. 5 is a first partial cross-sectional view of the invention.

FIG. 6 is a second partial cross-sectional view of the invention.

FIG. 7 is a first schematic view according to the preferred embodiment of the invention.

FIG. 8 is a second schematic view according to the preferred embodiment of the invention.

FIG. 9 is a third schematic view according to the preferred embodiment of the invention.

### DETAILED DESCRIPTION OF THE PREFERRED EMBODIMENTS

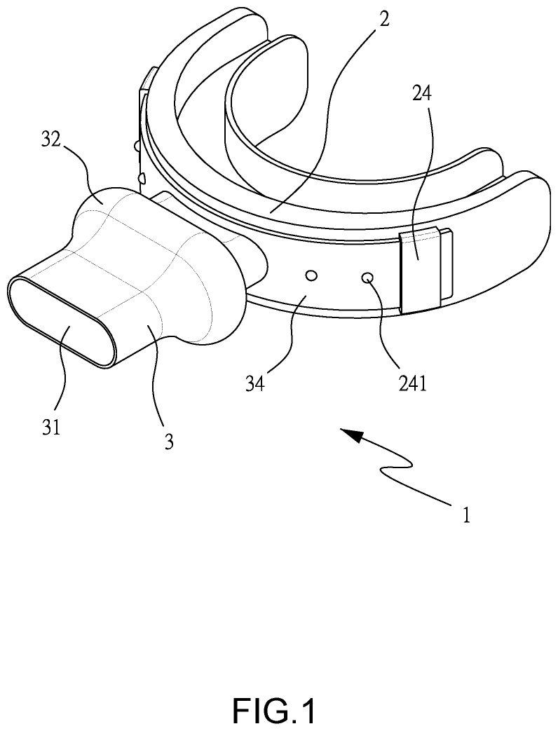

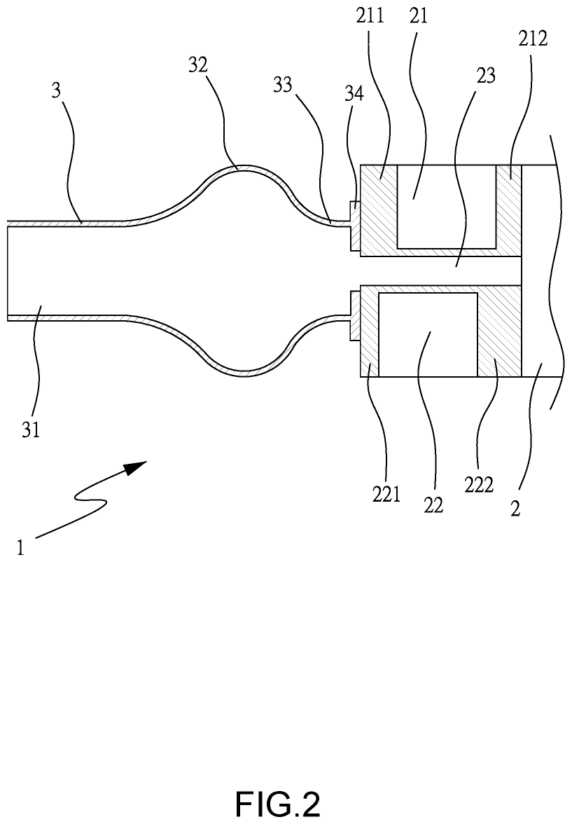

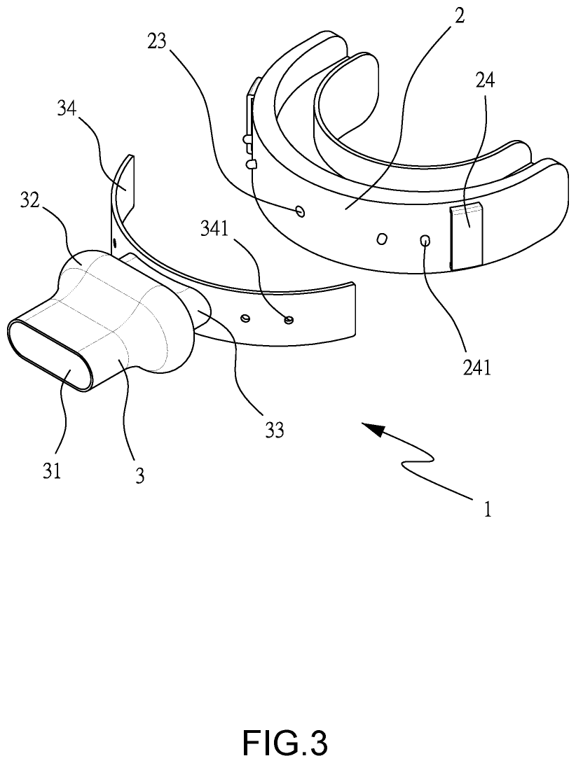

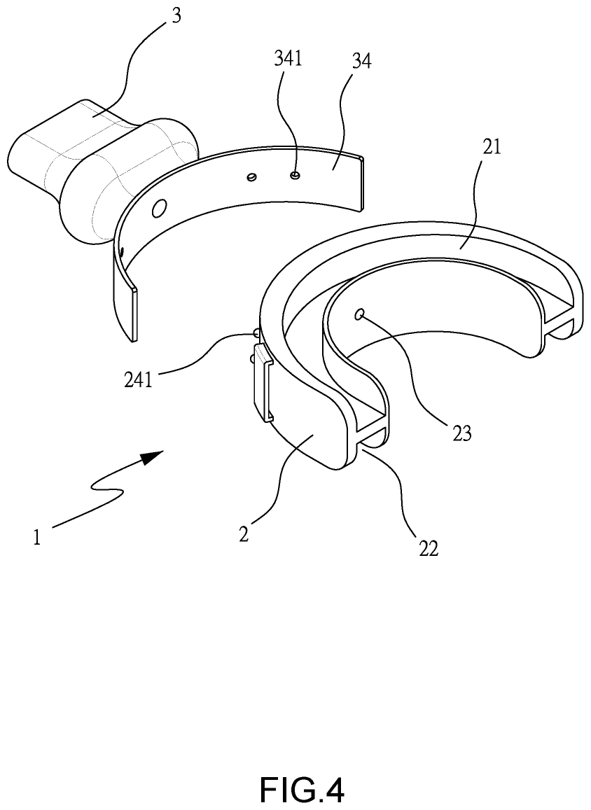

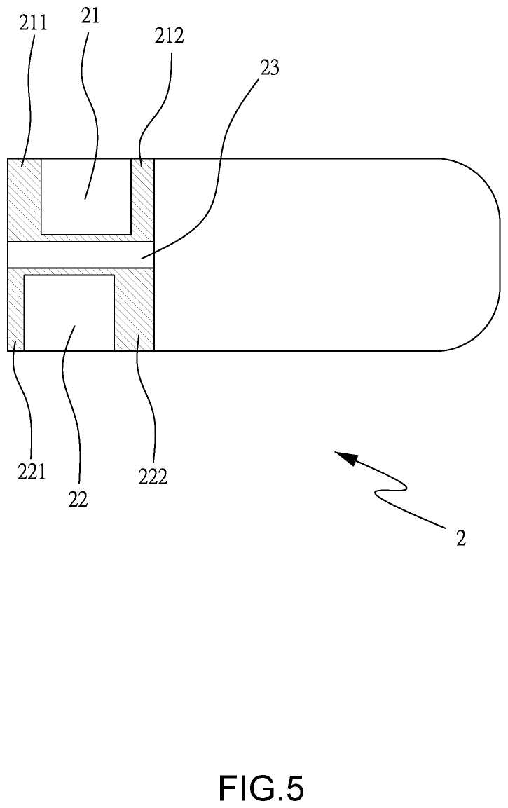

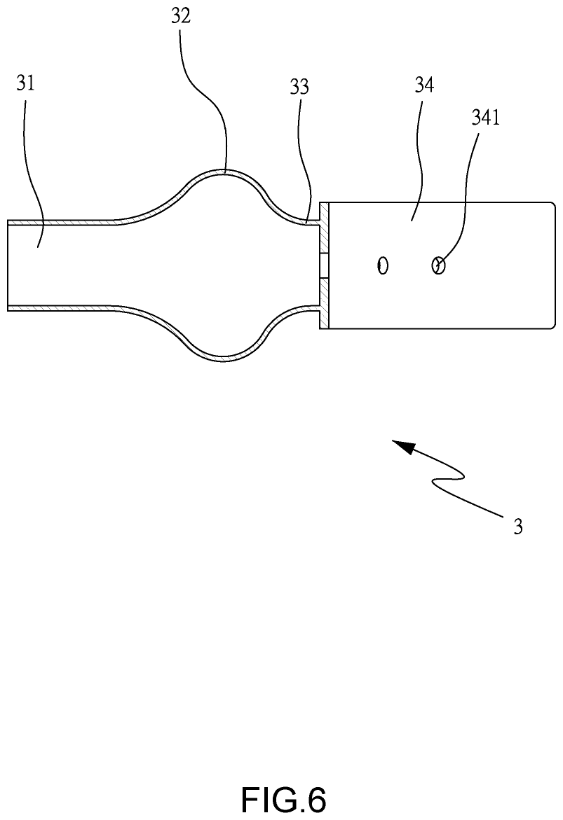

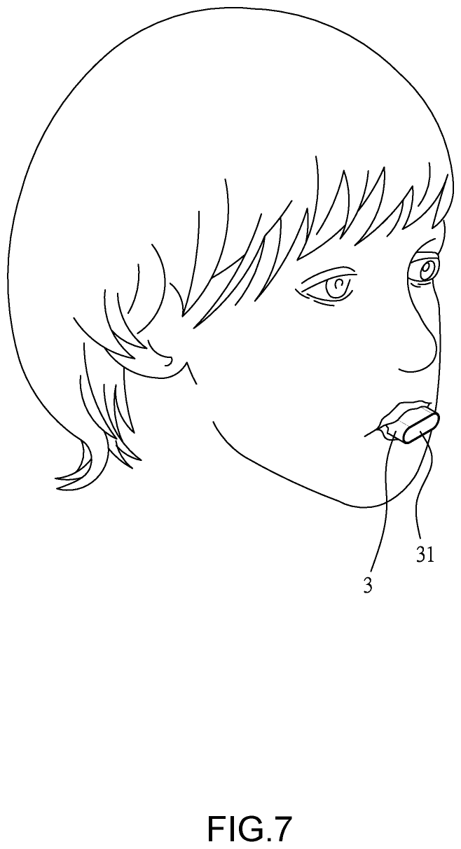

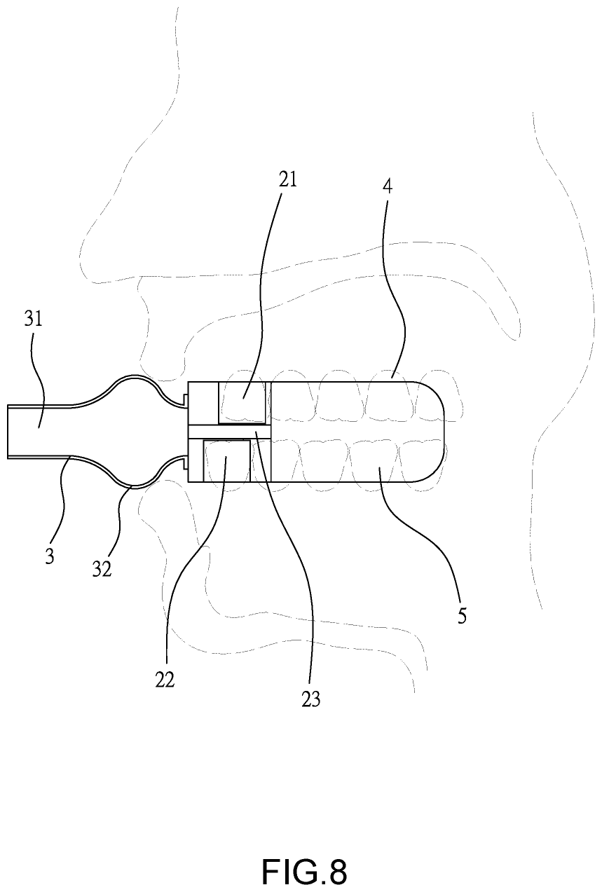

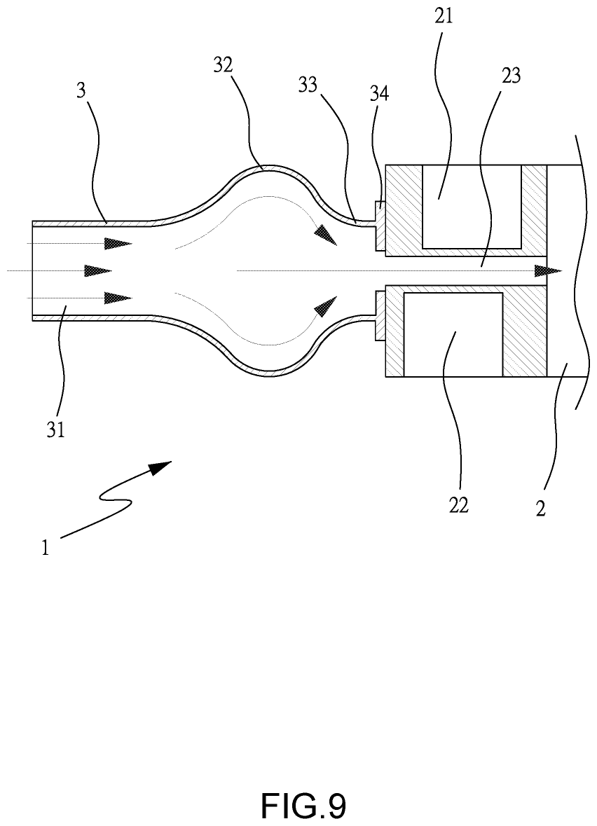

Referring to FIGS. 1 to 9, an anti-snoring device 1 of the invention mainly includes a base 2 and a flow-guide conduit 3.

The base 2 has an upper occlusal channel 21, a lower occlusal channel 22, an air intake hole 23 and an insert slot 24.

The flow-guide conduit 3 has an inlet port 31, a bump 32 and a rear end portion 33, and two corresponding convex sides 34 extended from the rear end portion 33. The two corresponding convex sides 34 are embedded into two insert slots 24 of the base 2.

The air intake hole 23 is disposed between the upper occlusal channel 21 and the lower occlusal channel 22, and entirely penetrates through the base 2.

A side edge of the insert slot 24 of the base 2 is additionally provided with multiple projections 241.

A front-to-rear position difference is present between the upper occlusal channel 21 and the lower occlusal channel 22.

The upper occlusal channel 21 has an upper front stop wall 211 and an upper rear stop wall 212.

The lower occlusal channel 22 has a lower front stop wall 221 and a lower rear stop wall 222.

The convex side 34 of the flow-guide conduit 3 is additionally provided with multiple through holes 341.

A dimension of an aperture of the inlet port 31 is smaller than a dimension of an aperture of the bump 32.

The dimension of the aperture of the inlet port 31 is greater than a dimension of an aperture of the air intake hole 23.

The flow-guide conduit 3 is integrally made of a flexible material.

Before use, the two corresponding convex sides 34 of the flow-guide conduit 3 are mainly embedded into the two insert slots 24 of the base 2. Upon use, the overall base 2 is placed within the oral cavity of the snoring patient, and the flow-guide conduit 3 is disposed outside the oral cavity. As shown in the drawings, the palate teeth 4 of the snoring patient mainly occlude the upper occlusal channel 21 of the base 2. Meanwhile, the mandible teeth 5 occlude the lower occlusal channel 22 of the base 2, and the flow-guide conduit 3 is disposed outside the oral cavity. When the snoring patient swallows the saliva, a negative pressure is generated in the oral cavity, and the air intake hole 23 of the base 2 generates a suction force by the negative pressure to suck the tongue frontward to obstruct the air intake hole 23, so that the snoring condition can be avoided.

As mentioned hereinabove, when the palate teeth 4 occlude the upper occlusal channel 21 of the base 2, the upper front stop wall 211 and the upper rear stop wall 212 of the upper occlusal channel 21 fix the palate teeth 4, and when the mandible teeth 5 occlude the lower occlusal channel 22 of the base 2, the lower front stop wall 221 and the lower rear stop wall 222 of the lower occlusal channel 22 fix the mandible teeth 5, so that the palate teeth 4 and the mandible teeth 5 can firmly occlude the base 2.

In addition, when the convex side 34 of the flow-guide conduit 3 is embedded into the insert slot 24 of the base 2, and the multiple projections 241 provided on the side edge of the insert slot 24 are embedded into the multiple through holes 341 provided on the convex side 34, the tight combination extent between both of the base 2 and flow-guide conduit 3 can be increased, and it is further possible to prevent the flow-guide conduit 3 from being detached from the base 2 upon use.

In addition, a front-to-rear position difference is present between the upper occlusal channel 21 and the lower occlusal channel 22. Thus, when the palate teeth 4 and the mandible teeth 5 occlude the base 2, a front-to-rear position difference is also generated between the palate and the mandible of the snoring patient. It mainly moves the mandible frontward through the front-to-rear position difference, so that smooth degree of breathing is enhanced.

In addition, the flow-guide conduit 3 is disposed at the front end of the base 2 for the additional purpose to function as a lip strength trainer to achieve the nasal breathing and mainly for the following reason. Because the dimension of the aperture of the bump 32 is greater than the dimension of the aperture of the inlet port 31, and the dimension of the aperture of the flow-guide conduit 3 is greater than the dimension of the aperture of the inlet port 31, the outside air enters from the inlet port 31 of the flow-guide conduit 3 and passes through the bump 32 and finally enters the oral cavity from the air intake hole 23 of the base 2 after the snoring patient has closed lips and swallowed the saliva. At this time, the extremely high negative pressure can be generated, so that the negative pressure sucks the tongue frontward to obstruct the air intake hole 23 of the base 2.

In addition, the overall flow-guide conduit 3 is made of the flexible material mainly because that the flow-guide conduit 3 still touches the lips of the snoring patient, and the flexible material can prevent the uncomfortable feeling of the lips.

In addition, when the anti-snoring device 1 needs to be cleaned, it is only necessary to extract the convex side 34 of the flow-guide conduit 3 from the insert slot 24 of the base 2, so that the base 2 and the flow-guide conduit 3 can be separated from each other rapidly to facilitate the cleaning. Alternatively, when the base 2 or the flow-guide conduit 3 is damaged, only one of them needs to be replaced.

New characteristics and advantages of the invention covered by this document have been set forth in the foregoing description. It is to be expressly understood, however, that the drawings are for the purpose of illustration only and are not intended as a definition of the limits of the invention. Changes in methods, shapes, structures or devices may be made in details without exceeding the scope of the invention by those who are skilled in the art. The scope of the invention is, of course, defined in the language in which the appended claims are expressed.

What is claimed is:

1. A brace-type anti-snoring device, comprising:
a base having an upper occlusal channel, a lower occlusal channel, an air intake hole and multiple insert slots; and
a flow-guide conduit having an inlet port, a bump, a rear end portion, and two corresponding convex sides extended from the rear end portion, wherein the two corresponding convex sides are embedded into two of the insert slots of the base;
wherein the base is mainly placed within an oral cavity, palate teeth occlude an upper occlusal channel of the base, and mandible teeth occlude a lower occlusal channel of the base while the flow-guide conduit is disposed outside the oral cavity, so that after a user has swallowed saliva, a negative pressure difference is generated in the oral cavity and a tongue is sucked frontward and obstructs the air intake hole of the base concurrently to achieve an objective of preventing snoring.

2. The brace-type anti-snoring device according to claim 1, wherein the air intake hole is disposed between the upper occlusal channel and the lower occlusal channel, and entirely penetrates through the base.

3. The brace-type anti-snoring device according to claim 1, wherein a side edge of the insert slot of the base is additionally provided with multiple projections.

4. The brace-type anti-snoring device according to claim 1, wherein a front-to-rear position difference is present between the upper occlusal channel and the lower occlusal channel.

5. The brace-type anti-snoring device according to claim 1, wherein the upper occlusal channel has an upper front stop wall and an upper rear stop wall.

6. The brace-type anti-snoring device according to claim 1, wherein the lower occlusal channel has a lower front stop wall and a lower rear stop wall.

7. The brace-type anti-snoring device according to claim 1, wherein the convex side of the flow-guide conduit is additionally provided with multiple through holes.

8. The brace-type anti-snoring device according to claim 1, wherein a dimension of an aperture of the inlet port is smaller than a dimension of an aperture of the bump.

9. The brace-type anti-snoring device according to claim 1, wherein a dimension of an aperture of the inlet port is greater than a dimension of an aperture of the air intake hole.

10. The brace-type anti-snoring device according to claim 1, wherein the flow-guide conduit is integrally made of a flexible material.
